(19) United States
(12) Patent Application Publication
(10) Pub. No.: US 2016/0242979 A1
(43) Pub. Date: Aug. 25, 2016

(54) METHOD AND APPARATUS FOR A CONTROLLED DISPENSING OF PARTICLES

(71) Applicant: Bluestone Technology GmbH, Worrstadt (DE)

(72) Inventors: FRANK GORGEN, Nieder-Olm (DE); TORSTEN SEHLINGER, UNDENHEIM (DE)

(73) Assignee: Bluestone Technology, GmbH, Worrstadt (DE)

(21) Appl. No.: 15/047,469

(22) Filed: Feb. 18, 2016

(30) Foreign Application Priority Data
Feb. 20, 2015 (DE) 102015102492.6

(51) Int. Cl. A61G 10/02; A61B 5/00; B05B 7/14

(52) U.S. Cl. CPC A61G 10/02; B05B 7/1486; A61B 5/411

(57) ABSTRACT

In a method and in an apparatus for a controlled dispensing of particles, allergenic particles of at least one first kind and one second kind are dispensed into a space, wherein the particles are dispensed into different corridors within the space which do not overlap.

The present invention relates to a method and to an apparatus for a controlled dispensing of allergenic particles to expose patients or subjects with allergies to a metered and controlled quantity of allergenic particles. Since the patient is exposed to different allergens in different concentrations, it is possible to discover which symptoms occur to which degree.

Various methods and apparatus for dispensing allergenic particles are known from the prior art in which the dispensing of the particles takes place via a turbulent air flow, which results in a discontinuous and spatially uneven distribution of the particles.

It is the object of the present invention to provide a method and an apparatus for a continuous dispensing of allergenic particles with which patents can be tested for different allergens in a short time.

This object is satisfied by the features of the independent claims.

In the method in accordance with the invention for a controlled dispensing of allergenic particles, allergenic parties of at least one first kind and one second kind are dispensed into one and the same space, wherein the allergenic particles of the first kind are dispensed into the space at a first dispensing point and the allergenic particles of the second kind are dispensed into the space at a second dispensing point, with the two dispensing points being spaced apart from one another. In accordance with the invention, the allergenic particles of the first kind are only dispensed into a first corridor and the allergenic particles of the second kind are only dispensed into a second corridor within one and the same space, wherein the two corridors do not overlap at least over a predefined extent region.

The method in accordance with the invention is based on the recognition that allergenic particles of different kinds can absolutely be dispensed in a directed manner within one and the same space such that they at least do not overlap within a specific extent region, starting from the respective dispensing point. An overlap of the corridors can take place outside the predefined extent region, which is not harmful if the inhalation zone of the patient or patients lies within the predefined extent region.

On the one hand, different patients can be exposed to different allergenic particles within one and the same space by the method in accordance with the invention. On the other hand, one and the same patient can be tested for different allergens within a very short time.

Advantageous embodiments of the invention are described in the description, in the drawing and in the dependent claims.

In accordance with a first advantageous embodiment, the corridors into which the different allergenic particles are dispensed are aligned downwardly in a vertical direction. A uniform dispensing of the particles within the associated corridor is hereby promoted. It can also be advantageous in this connection if the center axes of the corridors are aligned substantially in parallel with one another since this contributes to precluding as much as possible a mixing of the different particles.

It can be advantageous for carrying out the method in accordance with the invention if an air flow is produced at least in the region of the dispensing points in the space having a preferential direction which faces in the direction of gravity. In this manner, the allergenic particles are moved downwardly by the surrounding air flow after the dispensing into the space without any real transverse movement arising. It is advantageous in this respect if the air flow is produced as a substantially laminar air flow to avoid an unwanted mixing of the different particles.

To produce an air flow having a downwardly directed preferential direction, air can be introduced into the space by an air supply which extends in the region of the top of the space and which in particular extends along two parallel walls of the space. Air can furthermore be extracted from the space by a floor extractor which in particular extends along two parallel walls of the space. The most varied arrangements of air supply and air extractor over the area of the space are, however, generally possible as long as it is ensured that an air flow having a preferential direction in the direction of gravity is at least produced in the region of the dispensing devices.

The particles can be dispensed at a very low speed into their respective corridors so that they are only accelerated downwardly in the direction of the floor of the space by acceleration of gravity or by the air flow having a downward preferential direction. It can, however, also be advantageous to dispense the particles into their respective corridors in the direction of gravity at a speed which is higher than the speed of the air flow.

In accordance with a further aspect of the present invention, it relates to an apparatus for carrying out a method of the above-described method, wherein said apparatus comprises a chamber, which is in particular mobile, in whose interior a space is provided, wherein at least one first dispensing device for allergenic particles of a first kind and one second dispensing device for allergenic particles of a second kind are provided, with the two dispensing apparatus being spaced apart from one another. The dispensing devices are arranged in the region of a top of the chamber and are oriented such that the allergenic particles of the first kind are only dispensed into a first corridor and the allergenic particles of the second kind are only dispensed into a second corridor within the space, wherein the two corridors do not overlap at least over a predefined extent region.

It can also be advantageous here to provide an air supply in the apparatus in the region of a top of the space and to provide an air extractor in the region of the floor of the space, wherein the air supply and the air extractor are configured and positioned such that an air flow having a preferential direction facing in the direction of gravity is at least produced in the region of the dispensing devices.

Only embodiments having no more than two dispensing devices for different allergenic particles will be described in the following description. It is, however, understood that in accordance with the invention two or more dispensing devices for allergenic particles of different kinds can be provided.

The present invention will be described in the following purely by way of example with reference to an advantageous embodiment and to the enclosed drawings. There are shown:

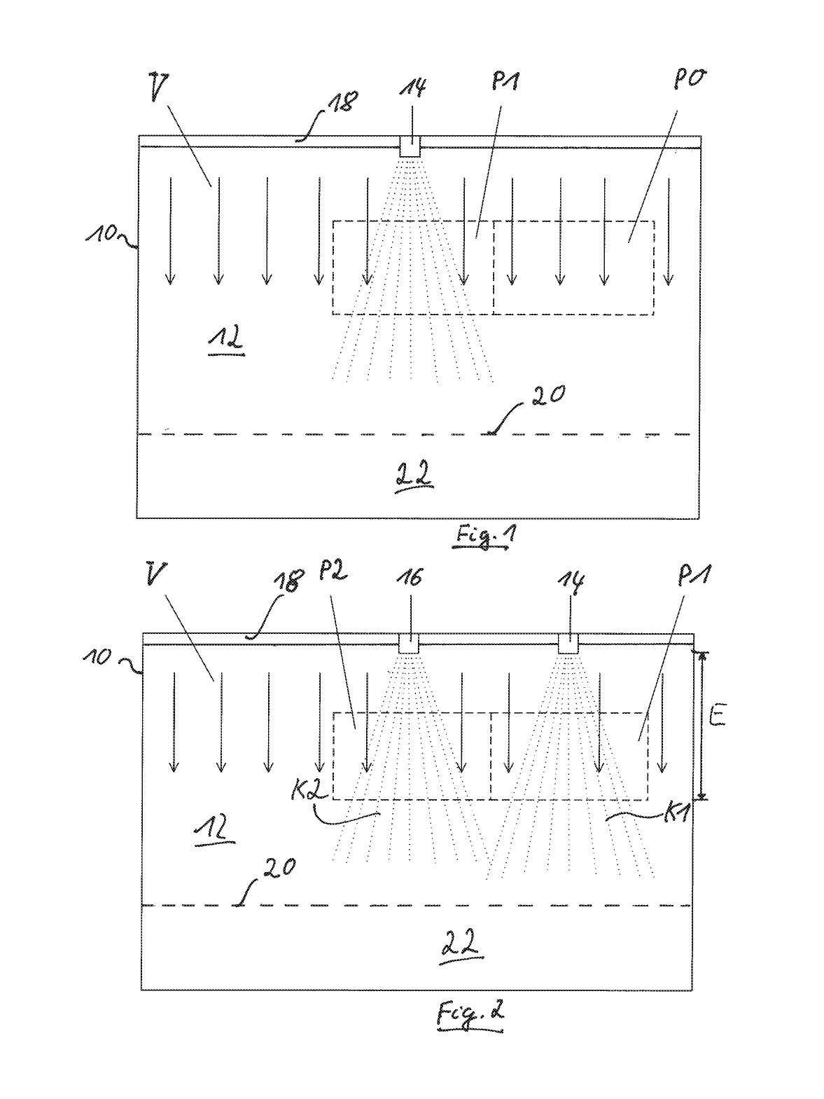

FIG. 1 a cross-section through a chamber; and

FIG. 2 a longitudinal section through the chamber of FIG. 1.

FIGS. 1 and 2 show in a very simplified schematic representation a cross-section and a longitudinal section through a chamber 10 which is in particular mobile, for example in the form of a container, and in whose interior a space 12 is provided. It is possible to walk into the space 12 and the latter has at least first dispensing device 14 for allergenic particles of a first kind and a second dispensing device 16 for allergenic particles of a second kind in the region of its top, with the two dispensing devices 14 and 16 being spaced apart from one another. The dispensing devices can be ones such as are described in German patent application DE 10 2014 116 694.9 whose content is incorporated by reference in the content of the present application and whose disclosure is included in the present application.

As FIG. 2 illustrates, the two dispensing devices 14 and 16 are arranged and oriented in the region of the top of the space such that the allergenic particles of the first kind are only dispenses into a first corridor K1 and the allergenic particles of a second kind are only dispenses by the second dispensing device 16 into a second corridor K2 within one and the same space 12. In the embodiment shown, the corridors are configured as conical starting from the dispensing devices and the two conical corridors K5 and K2 do not overlap over a predefined extent region E, starting from the dispensing devices 14 and 16. Nor are any dividing walls or the like provided between the corridors K1 and K2. In this respect, the extent region E is selected, starting from the dispensing devices, such that upper bodies of patients who are seated on predefined seats within the space 12 are located within the extent zone E. It is precluded in this manner that patients simultaneously inhale particles of different kinds. An overlap of the corridors K1 and K2 outside the predefined extent region E is not harmful in this regard since no inhalation by a patient takes place outside the extent region E.

To assist the dispensing of the allergenic particles only in their associated corridors K1 and K2, an air supply 18 is provided in the region of the top of the space 12 which is shown very schematically and which preferably at least produces an air flow in the region of the dispensing devices 14 and 16, and preferably in the total space region, which has a preferential direction V which faces in the direction of gravity. In this respect, the air supply 18 can generally be formed in different manners in the region of the top, for example by air ducts which are provided with air outlet openings.

A grid bottom 20 is provided in the space 12 for the extraction of the space air and an air extractor 22 is provided beneath it with which the space air and also the air provided with allergenic particles is extracted.

As FIG. 2 illustrates, the corridors K1 and K2 are aligned downwardly in the vertical direction, with the center axes of the corridors K1 and K2 being aligned substantially in parallel with one another. The air flow having a preferential air direction vertically downwardly, i.e. in the direction of gravity, is preferably produced as a substantially laminar air flow, with the air in particular being able to be introduced from the top along at least two parallel walls of the space and being able to be extracted from the bottom.

In accordance with the invention, a defined particle space P1 in which only articles of a first kind are located which have been dispensed from the first dispensing device 14 into the corridor K1 is created within one and the same space 12 using the described methods and using the described apparatus. In addition to this defined particle space P1, a space P0 guaranteed free of particles can be provided at one or more sides so that a patient can be exposed in one and the same space 12 in the particle space P1 while another patient is not exposed in the particle-free space P0.

It is simultaneously possible to provide, in addition to the particle space P1, a defined particle space P2 in which only allergenic particles of a second kind are located which have been dispensed by the second dispensing device 16 into the corridor K2. One or more specific regions with particles are thus present within one and the same space 12 without any dividing walls, separators or barriers, whereas there are specific regions in the same space 12 with either no particles or with different particles.

Generally any desired particles are suitable for use in the above-described methods, in particular allergenic particles, for example natural or pretreated pollen.

1. A method for a controlled dispensing of allergenic particles of at least one first kind and one second kind into a space, wherein the allergenic particles of the first kind are dispensed into the space at a first dispensing point and the allergenic particles of the second kind are dispensed into the space at a second dispensing point spaced apart from the first dispensing point, wherein the allergenic particles of the first kind are only dispensed into a first corridor and the allergenic particles of the second kind are only dispensed into a second corridor within the space, with the two corridors not overlapping at least over a predefined extent region.

2. A method in accordance with claim 1, wherein the corridors are aligned downwardly in a vertical direction.

3. A method in accordance with claim 1, wherein center axes of the corridors are aligned substantially in parallel with one another.

4. A method in accordance with claim 1, wherein an air flow having a preferential direction which faces in the direction of gravity is produced in the space at least in the region of the dispensing points.

5. A method in accordance with claim 4, wherein the air flow is produced as a substantially laminar air flow.

6. A method in accordance with claim 1, wherein air is extracted from the space by a floor extraction device.

7. A method in accordance with claim 6, wherein the floor extraction device extends at least along two parallel walls of the space.

8. A method in accordance with claim 1, wherein air is introduced into the space through an air supply.

9. A method in accordance with claim 8, wherein the air supply extends at least along two parallel walls of the space.

10. A method in accordance with claim 4, wherein the particles are dispensed in the direction of gravity into their corridors at a speed which is higher than the speed of the air flow.

11. An apparatus for carrying out a method for a controlled dispensing of allergenic particles of at least one first kind and one second kind into a space in accordance with at least one of the preceding claims, said apparatus comprising:
a chamber in whose interior the space is provided;
a first dispensing device for allergenic particles of the first kind and a second dispensing device for allergenic particles of the second kind which is spaced apart from the first dispensing device,
wherein the dispensing devices are arranged and are oriented in the region of a top of the space such that the allergenic particles of the first kind are only dispensed into a first corridor and the allergenic particles of the second kind are only dispensed into a second corridor within the space, wherein the two corridors do not overlap at least over a predefined extent region.

12. An apparatus in accordance with claim 11, wherein the chamber is a mobile chamber.

13. An apparatus in accordance with claim 11, wherein an air supply is provided which extends in the region of a top of the space and an air extractor is provided in the region of a floor of the space, wherein the air supply and the air extractor are configured and positioned such that an air flow is at least produced in the region of the dispensing devices having a preferential direction which faces in the direction of gravity.
